(19) United States
(12) Patent Application Publication
(10) Pub. No.: US 2018/0363020 A1
(43) Pub. Date: Dec. 20, 2018

(54) PATHOGEN DETECTION METHOD

(71) Applicant: NORTH-WEST UNIVERSITY, Potchefstroom (ZA)

(72) Inventors: Cornelius Johannes Francois TAUTE, Potchefstroom (ZA); Du Toit LOOTS, Potchefstroom (ZA)

(21) Appl. No.: 16/008,856

(22) Filed: Jun. 14, 2018

(30) Foreign Application Priority Data
Jun. 15, 2017 (NL) 2019074

(51) Int. Cl. C12Q 1/00

(52) U.S. Cl. CPC C12Q 1/008; G01N 2333/35

(57) ABSTRACT

The present invention provides a method of detecting a Mycobacterium-specific metabolite in the form of mycothiol in a biological sample in vitro, including the steps of preparing a reaction mixture by combining the biological sample with an enzymatic solution containing a reaction buffer, nicotinamide adenine dinucleotide (NAD) and a formaldehyde dependent mycothiol dehydrogenase (FD-MDH); allowing reduction of NAD in the reaction mixture by interaction of FD-MDH with a predetermined Mycobacterium-specific metabolite in the form of mycothiol if present in the biological sample; and detecting reduced NAD within the sample, indicative of the presence of mycothiol in the biological sample and thus of a Mycobacterium infection in the source of the biological sample.

### FIELD OF APPLICATION OF THE INVENTION

The present invention relates to a method and kit for the detection of a Mycobacterium-specific metabolite in a biological sample.

### BACKGROUND TO THE INVENTION

Mycobacterial disease and specifically tuberculosis (“TB”) associated with Mycobacterium tuberculosis, is responsible for well over a million deaths a year; with mortality being particularly associated with instances of HIV infection. Early detection and positive identification of Mycobacterium infection is a crucial component of treating the disease, as it can be transmitted by aerosol droplets and thus has high transmission rates in cases of active infection. Additionally, multiple drug resistant strains of M. tuberculosis; including Multiple Drug Resistant (MDR) strains; are presently known, requiring prompt identification to ensure that the correct course of treatment is followed.

The usual method for diagnosing Mycobacterium infection is by detection of acid-fast bacteria in sputum by direct microscopy and culturing. Although microscopy using Ziehl-Neelsen (ZN) staining for the detection of TB is fast and inexpensive, when done properly, it only detects 60 to 70% of all adults with pulmonary TB, as compared to sputum cultures. In practice, however, these figures are far lower. Culturing is currently classified as the “gold standard” for the diagnosing TB, and is currently recommended for that purpose in all developing countries. This, however, is also not without its limitations; the major one being the time taken for diagnosis, namely 2-8 weeks. Molecular biology techniques for genotyping bacterial content in sputum involve tedious sample preparation to break down the complex sputum microstructure as well as mycobacterium cell walls to obtain the mycobacterium genetic material, with polymerase chain reaction (PCR). More recently, PCR technology has been used for TB diagnostic applications. This however is also not without limitations considering its high costs and the need for high tech infrastructure and well-trained personnel. The high incidence of false-positive results due to laboratory cross-contamination also limits its performance under field conditions, and its sensitivity in smear negative—culture confirmed samples has been reported to be anything between 47-68%. PCR is an expensive, highly sensitive and accurate molecular biology tool, prone to sample handling errors and inhibitors from improper sample preparation, which excludes it from use in poor and uneducated areas, requiring trained personnel only. These inadequacies result in prolonged exposure of an untreated infectious individual to his/her community and are thought to be a major factor contributing to the increased prevalence of TB and MDR-TB infection, disease and mortality.

Early detection of tuberculosis, could lead to 1. The prevention of the spread thereof and the development of new strains of MDR-TB, 2. a reduced mortality and 3. More effective treatment outcomes. The development of rapid diagnostic point-of-care (POC) devices has received more attention by researchers in the past few years, specifically with the increased interest in nanotechnology and nanoparticles. Commercial POCs have good specificity (90-95%) but current limitations are poor detection limits and low to moderate sensitivity.

The class of thiols, known as mycothiols, have been recognised as being unique to the Actinomycetes (Gerald et al (1996) Journal of Bacteriology 178(7): 1990-1995), a class of organisms which including infectious TB causing mycobacteria, the latter of which are the only genus of Actinomycetes resulting in TB associated symptoms, hence it's specificity for identifying these organisms in TB patient sputum. This, in turn, has resulted in the development of a number of immunoassay systems for detecting mycothiols in biological samples (Unson et al (1998) Journal of Immunological Methods 214: 29-39 and Unson et al (1999) Journal of Clinical Microbiology 37(7): 2153-2157). These methods are capable of high sensitivity using enzyme-linked immunosorbent assay (ELISA) approaches, but however, requires long setup and processing steps (at least 10 hours) in order to achieve these results.

Given the above, it is clear that there exists a present need for a rapid, sensitive and highly specific assay for the detection of Mycobacterium infections in human subjects.

### OBJECT OF THE INVENTION

Accordingly, it is an object of the present invention to provide a method of detecting a Mycobacterium-specific metabolite in a biological sample with which the aforesaid disadvantages could be overcome or at least minimised.

### SUMMARY OF THE INVENTION

According to a first aspect thereof, there is provided a method of detecting a Mycobacterium-specific metabolite in the form of mycothiol in a biological sample in vitro, including the steps of:
preparing a reaction mixture by combining the biological sample with an enzymatic solution containing a reaction buffer, nicotinamide adenine dinucleotide (NAD) and a formaldehyde dependent mycothiol dehydrogenase (FD-MDH);
allowing reduction of NAD, to generate NADH, in the reaction mixture by interaction of FD-MDH with a predetermined Mycobacterium-specific metabolite in the form of mycothiol if present in the biological sample; and
detecting reduced NAD (NADH) within the sample, indicative of the presence of mycothiol in the biological sample and thus of a Mycobacterium infection in the source of the biological sample.

The Mycobacterium infection is typically, but not exclusively, an infection of Mycobacterium tuberculosis, or infection with other Mycobacterium species, which is typically be treated in the same manner.

Furthermore, according to the invention, the step of detecting reduced NAD (i.e. NADH) in the sample, includes the step of detecting the change through any one or more methods selected from the group consisting of colorimetric reactions, enzymatic assays, chromatographic methods, mass spectroscopy and spectrophotometric methods.

The biological sample may be selected from the group consisting of pure metabolites, cell extracts, blood, sputum, urine, cerebrospinal fluid, liquid cultures, and combinations thereof.

Further according to the invention, the enzymatic solution includes adjuncts drawn from the group consisting of salts, aldehydes, amines, hydroxides, reducing agents and combinations thereof.

Preferably, the enzymatic solution is selected from the group consisting of sodium chloride, formaldehyde, tris(hydroxymethyl)aminomethane, dithiothreitol, and combinations thereof.

The enzymatic reaction may be carried out at a temperature of from 25 to 40 degrees Celsius and for a period of from 2 to 840 minutes. Alternatively, the enzymatic reaction is carried out for a period of from 2 to 80 minutes. Further alternatively the enzymatic reaction may be carried out for a period of from 2 to 20 minutes.

The enzymatic reaction may be carried out in a cuvettes, clear multiwell-plate, or glass sample vials, with the total volume of the individual reactions being under 250 uL. Alternatively, the reaction is carried out in conventional laboratory glassware.

According to a second aspect of the invention, there is provided a kit for detecting a Mycobacterium-specific metabolite in a biological sample in vitro, comprising an enzymatic solution containing a reaction buffer, nicotinamide adenine dinucleotide (NAD) and a formaldehyde dependent mycothiol dehydrogenase (FD-MDH); and a suitable container for receiving a biological sample and the enzymatic solution to form a reaction mixture.

These and other objects, features and advantages of the invention will become apparent to those skilled in the art following the detailed description of the invention.

### BRIEF DESCRIPTION OF THE DRAWINGS

Preferred embodiments of the invention are described below with reference to the accompanying figures, wherein:

FIG. 1 is a graph depicting the standardization of the enzymatic reaction of MSH and MD-FaIDH in Tris-buffer where an increase in optical density is linearly related to an increase in NADH measured at 340 nm;

FIG. 2 is a graph depicting the standardization of the enzymatic reaction of MSH and MD-FaIDH to use a futile cycle in the presence of low concentrations of MSH and MD-FaIDH in Tris-buffer over 14 h, with (a), being the application of the assay to cultured cell lysates over for 14 h (b) and an enlarged view demonstrating the lower detection limit to be 0.05 ug (or 50 ng) cell extract equivalent. An increase in optical density is linearly related to an increase in NADH, measured at 340 nm; and

FIG. 3 is a graph depicting (a) the increase in optical density at 340 nm for the MSH-MD-FaIDH assay using sputum samples (culture positive; smear microscopy positive (C+ve; SM+ve); culture positive and smear microscopy negative (C+ve; SM−ve)) and TB negative sputum samples using culture and smear (sputum −ve) and (b) an enlarged area of the graph indicating a detectable signal generated for the TB+ samples with no perceptible signal for the controls or TB− samples (b).

The presently disclosed subject matter will now be described in greater detail hereinafter with reference to the accompanying examples, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

### DESCRIPTION OF PREFERRED EMBODIMENTS OF THE INVENTION

A method according to a preferred embodiment of the invention, for detecting a Mycobacterium-specific metabolite in the form of mycothiol in a biological sample in vitro includes the steps of:
preparing a reaction mixture by combining the biological sample with an enzymatic solution containing a reaction buffer, nicotinamide adenine dinucleotide (NAD) and a formaldehyde dependent mycothiol dehydrogenase (FD-MDH);
allowing reduction of NAD, to generate NADH, in the reaction mixture by interaction of FD-MDH with a predetermined Mycobacterium-specific metabolite in the form of mycothiol if present in the biological sample; and
detecting reduced NAD (NADH) within the sample, indicative of the presence of mycothiol in the biological sample and thus of a Mycobacterium infection in the source of the biological sample.

The method is particularly suitable, without limitation, for detecting Mycobacterium infection including that of Mycobacterium tuberculosis, but it is foreseen that the presence of any Actinomycetes having a distribution of mycothiol could be detected in a biological sample using the method according to the invention. However, considering the patient sample will be collected on the basis of symptoms associated exclusively to that caused by infectious mycobacteria, it would be specific for tuberculosis.

The biological sample is selected from the group consisting of pure metabolites, cell extracts, blood, sputum, urine, cerebrospinal fluid, liquid cultures, and combinations thereof.

The step of detecting reduced NAD (i.e. NADH) in the sample, includes detecting the change through any one or more methods selected from the group consisting of colorimetric reactions, enzymatic assays, chromatographic methods, mass spectroscopy and spectrophotometric methods.

The enzymatic solution is selected from the group consisting of salts and buffers, in particular, aldehydes, amines, hydroxides, reducing agents and combinations thereof. Preferably, the enzymatic solution comprises a mixture of sodium chloride, formaldehyde, tris(hydroxymethyl)aminomethane, dithiothreitol.

It may be appreciated that the rate of the enzymatic reaction is dependent on the starting concentration of the Mycobacterium-specific metabolite in the biological sample. Accordingly, the time taken to generate a certain concentration of NADH during the reaction will also be dependent upon the starting concentration of the Mycobacterium-specific metabolite in the biological sample.

The enzymatic reaction is carried out at a temperature of from 25 to 40 degrees Celsius and for a period of from 2 to 840 minutes. Alternatively, the enzymatic reaction is carried out for a period of from 2 to 80 minutes. Further alternatively the enzymatic reaction is carried out for a period of from 2 to 20 minutes.

It may be appreciated that the enzymatic reaction can be carried out in a number of apparatuses and volumes, with various options being well known to persons skilled in the art. In an embodiment of the invention, the enzymatic reaction is carried out in a cuvette, clear multiwell-plate, or small glass sample vials, with the total volume of the individual reactions being under 250 uL. In an alternative embodiment of the invention, the reaction is carried out in conventional laboratory glassware.

According to a second aspect of the invention, there is provided a kit for detecting a Mycobacterium-specific metabolite in a biological sample in vitro, comprising an enzymatic solution containing a reaction buffer, nicotinamide adenine dinucleotide (NAD) and a formaldehyde dependent mycothiol dehydrogenase (FD-MDH); and a suitable container for receiving a biological sample and the enzymatic solution to form a reaction mixture.

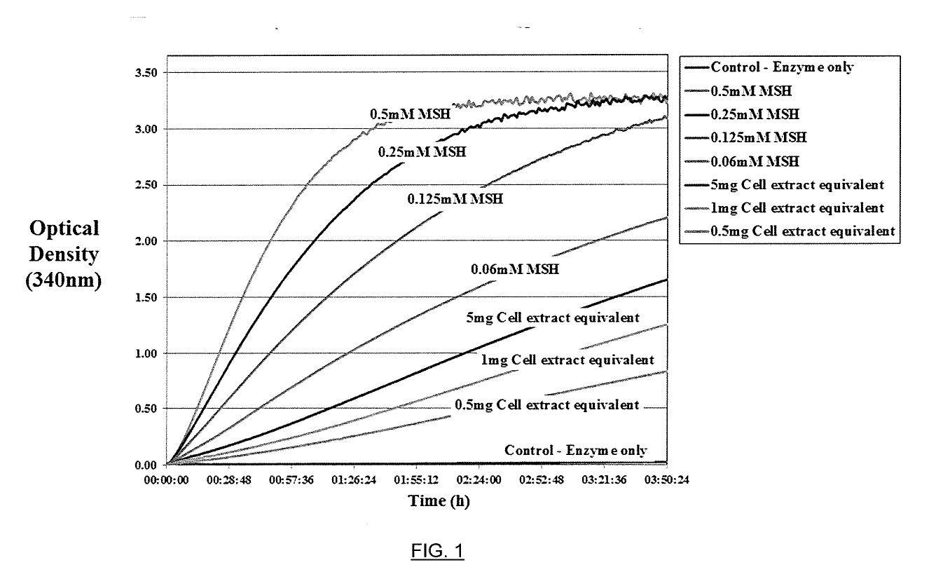

A non-limiting example of a preferred embodiment of the invention is described in more detail below, with reference to FIGS. 1 to 3.

### Example: A Visual Assay for the Detection of M. tuberculosis

According to a preferred embodiment of the present invention, a formaldehyde dependent mycothiol dehydrogenase (FD-MDH) is used to generate a reduced energy carrier (NADH) in the presence of formaldehyde adducts of mycothiol (MSH). The enzymatic solution buffer is dependent on pH, temperature, mycothiol (reduced) content, as well as formaldehyde content.

The assay may be performed in a high-throughput 96-well plate format, with reaction volume a total of 200 uL. The reaction takes 2 min to 14 hours at 30 degrees Celsius.

Standardization of the Enzymatic Assay

For the purposes of standardisation, pure synthetic mycothiol (trifluoroacetic acid salt) is used. This is made up to 10 mM stocks without extraction, as the trifluoroacetic acid salt prevents auto-oxidation of mycothiol. The MD-FaIDH activity screen is set up in a 96-well microplate (the general layout of which can be seen in Table 1). A pre-mixture is made containing all the components of the enzyme activity screen (Lessmeier et al; 2013). The premix composition consists of 100 mM Tris (pH 8.25), 500 mM NaCl, 5 mM NAD⁺, 1 mM DTT, 1-5 mM Formaldehyde, and 0.001-0.05 mg/mL MD-FaIDH. The “sample” would contain a mixture of ingredients to replicate the consistence of the actual patient sample material used, or contain actual patient sample matrix devoid of the infectious pathogen and/or the biological compound being detected (in this instance mycothiol).

TABLE 1
An example of the general layout and preparation for standardising of
the proposed mycothiol assay and preliminary cell extract tests using H37Rv
M. tuberculosis isolated from liquid cultures

| | | Assay | | | | | | | Sample |
|---|---|---|---|---|---|---|---|---|---|
| | Blank | Control | Standard 1 | Standard 2 | Standard 3 | Standard 4 | Sample 1 | Sample 2 | Control |
| Premix | 0 | 150 | 150 | 150 | 150 | 150 | 150 | 150 | 150 |
| (μL) | | | | | | | | | |
| MSH | 0 | 0 | 0.5 | 0.25 | 0.125 | 0.06 | 0 | 0 | 0 |
| (mM) | | | | | | | | | |
| Sample | 0 | 0 | 0 | 0 | 0 | 0 | 20 | 20 | 0 |
| (μL) | | | | | | | | | |
| ddH₂O | 150 | 0 | 50 | 50 | 50 | 50 | 30 | 30 | 50 |
| (μL) | | | | | | | | | |

| | |
|---|---|
| TOTAL | 200 μL |
| volume | |

The increase in optical density is measured at 340 nm (NADH) on a BioTEK Synergy UV-Vis spectrometer plate reader, or any similar model instrument, with the incubator temperature set at 30 degrees Celsius. All blank and wavelength corrections were done using the Gen5 software package. The kinetic interval is read at 30-90 s intervals, dependent on the number of samples. For all kinetic assays, pure MSH is used as a standard for increases in optical density (340 nm). All readings are done in triplicate. For all purposes, where possible, MSH standards where dissolved in the same matrix as that of the pathogen containing samples, e.g. liquid culture growth media or extraction buffer for the case of sputum samples.

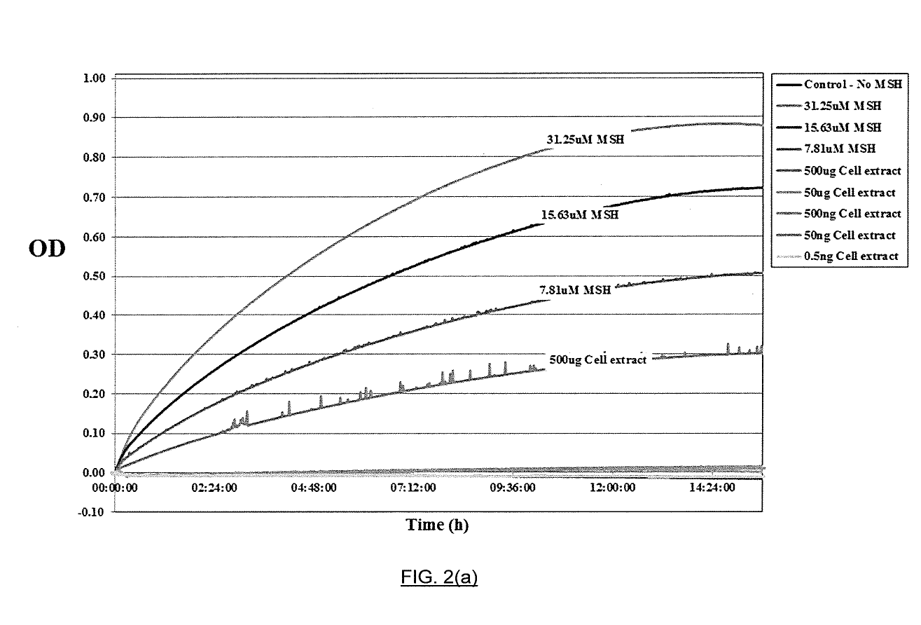

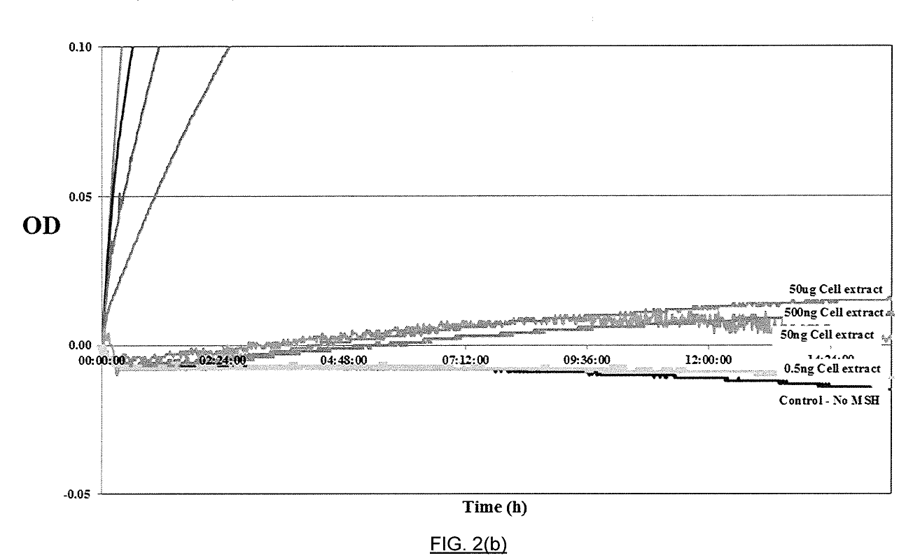

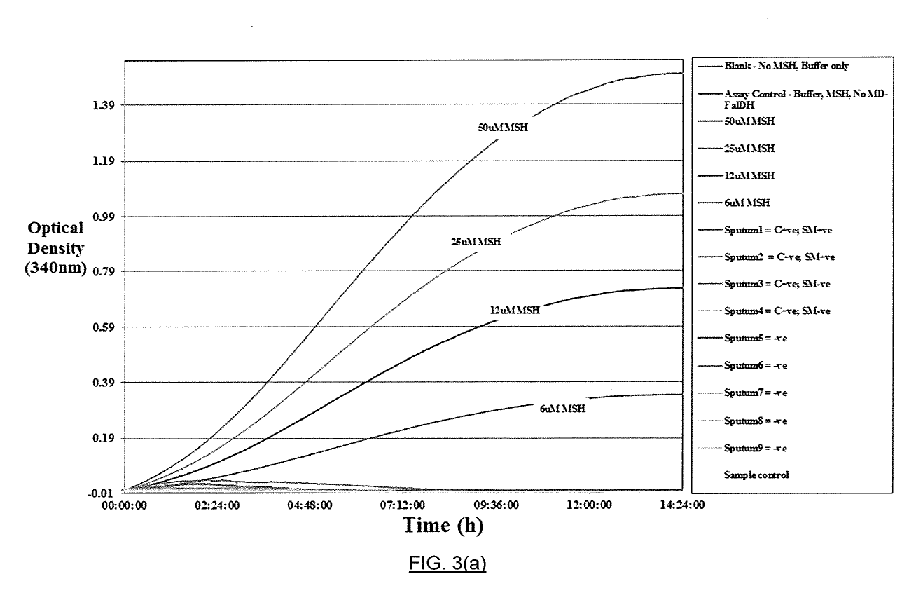

For the purposes of this assay, the absence of NaCl or substituting the Tris with HEPES/MOPS buffer has no significant effect on the increase in optical density at 340 nm over time. The determining factor was found to be solely pH dependent, where optimal activity corresponds to that described by Lessmeier et al (2013) in the literature (pH 8.25). The increase in optical density at 340 nm (NADH) can be seen in FIG. 1 and FIG. 2a, where, in the case of the standards, the increase of optical density is of a mixture containing pure mycothiol over 4 h and 14 h as described previously, respectively. The composition of the standards used for the results generated in FIG. 1, included using 0.05 mg/mL MD-FaIDH with a MSH standard series of: 0.5 mM, 0.25 mM, 0.125 mM, and 0.06 mM respectively. The composition of the standards used for the results generated in FIG. 2a, included using 0.002 mg/mL MD-FaIDH with a MSH standard series of: 0.032 mM, 0.016 mM, and 0.008 mM.

The increase in optical density at 340 nm (NADH) from the enzymatic reaction done on liquid cultured H37Rv M. tuberculosis cell lysates can be seen in FIG. 1 and FIG. 2b, for 4 hours and 14 hours respectively, are depicted and labelled as 0.5, 1 and 5 mg cell extract equivalents). Sample control refers here to a water/matrix sample which went through the same preparation steps as the samples, which contains no mycothiol or enzyme.

For cell extracts (lysates) from liquid cultured M. tuberculosis, a new extraction buffer was developed. The buffer is compiled based on numerous protocols, specifically protocols for the isolation of genomic DNA from plant materials. The buffer appears white-cloudy until it is in the presence of cell material, where the solution becomes optically clear. The buffer (pH 8) consists of 100 mM 4-(2-hydroxyethyl)-1-piperazineethanesulfonic acid) (HEPES) buffer, 500 mM NaCl, 5% sodium dodecyl sulphate (SDS), 5% cetyltrimethylammonium bromide (CTAB). The following is a general protocol for using the lysis buffer:

An excess amount of lysis buffer (100-500 μL) is added to the 50 mg of the M. tuberculosis pelleted wet cell mass isolated from liquid cultures. The cell pellet is mechanically disrupted through repetitive micro-pipetting in the same vial until particulates are visible and the sample has a creamy texture and appearance. The cell pellet-lysis buffer mixture is then vigorously vortexed for 60 s, followed by heating at 65 degrees Celsius for 30-45 minutes or at 90 degrees Celsius for 20 minutes. A final vortex step is done for 10 s. The cell debris is sedimented via centrifugation (at the a relative centrifugal force (RCF) of 4000 g) and the supernatant is used for subsequent analysis. Alternately the samples will sediment by lowering temperature of the mixture (e.g. by placing it on ice or in a refrigerator or freezer) or via gravity sedimentation.

Table 2 represents a typical experimental protocol making use of the lysis buffer described herein above.

TABLE 2
An example of the general layout and preparation for the determining
the limits of detection of the proposed mycothiol assay using cells
isolated fromH37Rv M. tuberculosis liquid cultures.

| | | Assay | | | | | | Sample |
|---|---|---|---|---|---|---|---|---|
| | Blank | Control | Standard 1 | Standard 2 | Standard 3 | Sample 1 | Sample 2 | control |
| Premix (μL) | 0 | 150 | 150 | 150 | 150 | 150 | 150 | 150 |
| MSH (mM) | 0 | 0 | 0.032 | 0.016 | 0.008 | 0 | 0 | 0 |
| Sample (μL) | 0 | 0 | 0 | 0 | 0 | 20 | 20 | 0 |
| ddH₂O (μL) | 150 | 0 | 50 | 50 | 50 | 30 | 30 | 50 |

| | |
|---|---|
| TOTAL | 200 μL |
| volume | |

In the example below, for the purpose of determining the lower limit of detection (i.e. the minimum number of mycobacteria require to be present in a sample), 5 mg of wet cell mass was suspended in 500 μL lysis buffer and processed as described above. The cell lysate was then diluted to create dilution series equivalent to: 5 mg, 1 mg, 0.05 mg, 50 μg, 5 μg, 0.5 μg, 0.05 μg, 0.005 μg of wet cells, and 20 μL of each was used to prepare analytical samples as indicated in the sample column in Table 2. This experiment was repeated three times with each sample being read in triplicated, and similar results were obtained for each repeat the results of which are presented in FIG. 1 (for the 5 mg, 1 mg and 0.5 mg cell mass equivalents) and FIGS. 2a and b (500 μg-0.005 μg (or 0.5 ng).

TABLE 3
Estimated amount of mycobacteria cell equivalents per assay sample.
The calculations are based on the literature accepted value of 1 × 10⁸
cells per 3 mg wet cell paste.

| | Mass of wet cell paste equivalents* | | | | | | | |
|---|---|---|---|---|---|---|---|---|
| | 5 mg | 1 mg | 0.05 mg | 50 μg | 5 μg | 0.5 μg | 0.05 μg | 0.005 μg |
| Estimated | 1.6 × 10⁸ | 3.33 × 10⁷ | 1.6 × 10⁷ | 1.6 × 10⁶ | 1.6 × 10⁵ | 1.6 × 10⁴ | 1.6 × 10³ | 1.6 × 10² |
| amount of | | | | | | | | |
| CFU | | | | | | | | |

CFU = Cell Forming Units

The estimated detection sensitivities of the enzymatic assay, as summarised in Table 3, demonstrate an increase in optical density is observed in amounts as low as 0.05 μg (50 ng) equivalent cell lysate (FIG. 2b).

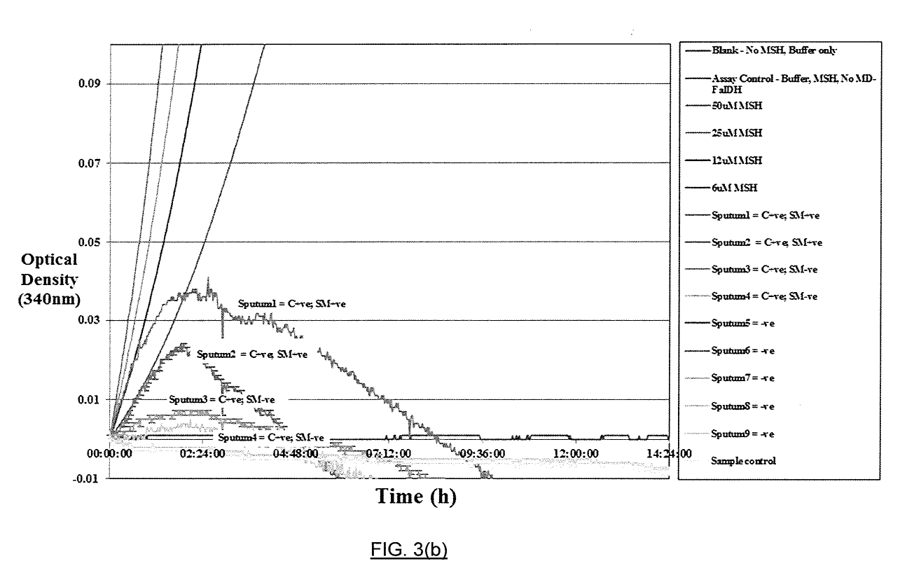

Subsequently, in order to assess the capacity of the enzymatic assay on detecting the presence of mycothiol in mycobacteria containing patient collected diagnostic samples, same extraction protocol as for liquid culture cell lysates were performed on patient collected sputum as an example as indicated in Table 4. The increase in optical density (340 nm) shows detectable signals for all 4 TB+ samples (including culture positive; smear microscopy positive (C+ve; SM+ve); culture positive and smear microscopy negative (C+ve; SM-ve)) and TB negative sputum samples, with no activity for the controls or 5 TB-ve samples (confirmed using cultures and smear microscopy (sputum −ve)) (FIG. 3). An enlargement of a specific area of the graph in FIG. 3a (FIG. 3b) shows a rapid increase in the optical density with a fast drop-off. This can possibly be attributed to remnant enzymatic activity from the sputum sample or small molecules interacting with the NADH. The sample control (FIG. 3a) underwent the exact same preparatory steps as the sputum samples. The increase in optical density at 340 nm for the 4 TB (+) samples were low but still detectable compared to the 5 TB (−) samples as well as the sample control (FIG. 3b). An attempt can be made to generate a result, similar to the cell lysates by optimizing the cell lysis protocol, the enzyme buffer system or incorporate enzymatic “enhancers” such as p-iodonitrotetrazolium violet or phenazine methosulfate (Hinman and Blass, 1981), as well as fluorescent reporter molecules, in order to further lower the detection limits (i.e. increase sensitivity of the method).

TABLE 4
An example of the general layout and preparation for the detection of
mycothiol in TB+ve and TB−ve sputum samples

| | | Assay | | | | | | | Sample |
|---|---|---|---|---|---|---|---|---|---|
| | Blank | Control | Standard 1 | Standard 2 | Standard 3 | Standard 4 | Sample 1 | Sample 2 | Control |
| Premix | 0 | 150 | 150 | 150 | 150 | 150 | 150 | 150 | 150 |
| (μL) | | | | | | | | | |
| MSH | 0 | 0 | 0.050 | 0.025 | 0.012 | 0.006 | 0 | 0 | 0 |
| (mM) | | | | | | | | | |
| Sample | 0 | 0 | 0 | 0 | 0 | 0 | 20 | 20 | 0 |
| (μL) | | | | | | | | | |
| ddH₂O | 150 | 0 | 50 | 50 | 50 | 50 | 30 | 30 | 50 |
| (μL) | | | | | | | | | |

| | |
|---|---|
| TOTAL | 200 μL |
| volume | |

During validation, the MSH-MD-FaIDH enzymatic assay for the detection of NADH directly from sputum extracted samples demonstrated a successful discrimination of all TB +ve (of which 2 where culture positive and smear positive and 2 culture positive and smear negative) and 5 TB −ve samples (FIG. 3).

It may be readily appreciated that the enzymatic component of the assay can be used for liquid cultures as well as sputum samples with further validation, and may be used in research as well as diagnostics.

The results indicate that the present invention is sensitive enough to detect 1.6×10⁷ 10⁴ CFU after 10 minutes incubation, and 1.6×10⁴ CFU after about 2 and a half hours and 1.6×10³ after approximately 6 hours. Considering this sensitivity and time to diagnosis, it makes it a good case for an alternative method to smear microscopy (requires 1×10⁴). However, when tested on actual patient collected sputum, this method was able to positively identify all TB positive samples, even those missed by conventional smear microscopy, suggesting that it is more sensitive than smear microscopy and most likely detects exogenous mycothiol produced by the Mycobacterium complex in the sputum matrix, which makes it a likely candidate for being as sensitive as culturing, which can only be confirmed in a bigger sample cohort.

A method according to the preferred embodiment of the invention, as described herein above, has a number of advantages over the prior art methods.

Firstly, the invention is simple to perform and can be commercialised at far lower costs than other existing high-sensitivity assay approaches. Secondly the test, as described herein above is comparatively faster than other methods; i.e. requiring at most a few hours to be completed, for multiple samples (when done using 96 well plates) simultaneously, and can be fully automated. This compares favourably, considering that other widely based TB diagnostic approaches are largely are timely, the fastest currently being PCR-based approaches, which require at least a few hours for the amplification step alone and may effectively take days to complete, and the fastest culturing methods 2 days at best (2-8 weeks on average). Smear microscopy, apart from having a far lower sensitivity when compared to the assay described here, is also labour intensive, requiring skilled histology training, potentially incorporating multiple handling errors and visual assessment which can introduce confirmation bias, typically taking several hours of preparation and analysis; however, and multiple sample preparation is a limiting factor time-wise.

Lastly, the test as described herein detects a metabolic marker, and thus does not rely on genetic material. This improves the accuracy of the present invention when compared to existing genetic approaches, as residual genetic material may produce false positive results.

Furthermore, this assay can be built into a point of care diagnostic device.

It will be appreciated that variations in detail are possible with a method according to the invention without departing from the scope of the appended claims.

What is claimed is:

1. A method of detecting a Mycobacterium-specific metabolite in the form of mycothiol in a biological sample in vitro, including the steps of:
preparing a reaction mixture by combining the biological sample with an enzymatic solution containing a reaction buffer, nicotinamide adenine dinucleotide (NAD) and a formaldehyde dependent mycothiol dehydrogenase (FD-MDH);
allowing reduction of NAD, to generate NADH, in the reaction mixture by interaction of FD-MDH with a predetermined Mycobacterium-specific metabolite in the form of mycothiol if present in the biological sample; and
detecting reduced NAD (NADH) within the sample, indicative of the presence of mycothiol in the biological sample and thus of a Mycobacterium infection in the source of the biological sample.

2. The method of claim 1, wherein the mycothiol is produced by M. tuberculosis.

3. The method of either claim 1, wherein the biological sample is selected from the group consisting of blood, pure metabolites, cell extracts, sputum, urine, cerebral spinal fluid, liquid cultures and combinations thereof.

4. The method of claim 1, wherein the enzymatic solution includes adjuncts drawn from the group consisting of salts, aldehydes, amines, hydroxides, reducing agents and combinations thereof.

5. The method of claim 4, wherein the enzymatic solution is selected from the group consisting of sodium chloride, formaldehyde, tris(hydroxymethyl)aminomethane, dithiothreitol, and combinations thereof.

6. The method of claim 1, wherein the reaction is carried out at a temperature of from 25 to 40 degrees Celsius and for a period of from 2 to 840 minutes.

7. The method of claim 1, wherein the NAD and/or reduced NAD concentration is measured by means of methods drawn from the group including colorimetric assays, enzymatic assays, chromatographic assays, mass spectroscopy and spectrophotometric assays, lateral flow device, naked eye detection, urine test strip and combinations thereof.

8. The method of claim 1, substantially as herein described and exemplified, and/or described with reference to the accompanying figures.
